(19) United States
(12) Patent Application Publication
(10) Pub. No.: US 2005/0075520 A1
(43) Pub. Date: Apr. 7, 2005

(54) System and method of producing bisphenol-A (BPA) using two stage crystallization

(76) Inventors: Drow Lionel O'Young, West Covina, CA (US); Shan Tao Hsieh, Princeton Junction, NJ (US); Vaibhav Kelkar, West Covina, CA (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 10/665,705

(22) Filed: Sep. 17, 2003

Related U.S. Application Data
(63) Continuation-in-part of application No. 10/632,664, filed on Aug. 1, 2003.
Application No. 10/632,664 is a continuation-in-part of application No. 10/099,227, filed on Mar. 13, 2002.

(51) Int. Cl. C07C 39/16

(52) U.S. Cl. 568/728

(57) ABSTRACT

A system and method are provided wherein the phase equilibrium of a system comprised of Phenol, Bisphenol-A and a solvent mixture comprising Acetone and Water is selectively controlled such that either crystalline Bisphenol-A, or an adduct of Bisphenol-A and Phenol is crystallized from the product solution. The present invention provides for the recovery of pure Bisphenol-A (BPA) by direct crystallization from solution.

### RELATED APPLICATIONS

This patent application is a continuation-in-part application of pending U.S. patent application Ser. No. 10/632,664 filed on Aug. 1, 2003 (Our file no. A-71205), which is a continuation-in-part application of pending U.S. patent application Ser. No. 10/099,227 filed on Mar. 13, 2002 (Our file no. A-71155), the entire disclosure of both are hereby expressly incorporated by reference herein.

### FIELD OF THE INVENTION

The present invention provides a system and method for production of Bisphenol-A (BPA). More specifically, the present invention provides a system and method for producing Bisphenol-A wherein certain phase equilibrium conditions are manipulated to recover substantially solid pure Bisphenol-A using two stage crystallization.

### BACKGROUND OF THE INVENTION

The production of bisphenols such as 2,2-bis (4-hydroxyphenyl)propane (Bisphenol-A, hereinafter sometimes referred to as “4,4-BPA” or simply identified as “BPA”) is an important process as Bisphenol-A is used as a feedstock or intermediate for the production of various polymers such as epoxy resins and polycarbonates. In one application, Bisphenol-A is reacted with phosgene to produce commercial polycarbonate resins. High quality polycarbonates, such as those used as optical media in the electronics and disk drive industry, require highly pure Bisphenol-A as a feedstock. Consequently, much effort has been focused on developing processes to produce Bisphenol-A of high purity.

In general, Bisphenol-A is produced by a well known liquid-phase condensation reaction of Phenol with Acetone in the presence of an acid catalyst such as hydrochloric acid, or more commonly, an acidic ion exchange resin as catalyst. The reaction product typically includes the desired Bisphenol-A, unreacted reactants, by-products of the reaction most notably Water, and a variety of impurities including isomers, analogs and homologs of Bisphenol-A. These include 2-(2-hydroxyphenyl)-2-(4-hydroxyphenyl)propane (hereafter referred to as the o,p-bisphenol isomer or “2,4-BPA”), dianins compound, chromans, trisphenols, polyphenols and unfavorably colored substances. A variety of processes are used to purify and recover Bisphenol-A crystals from the reaction product. Purification and recovery of the Bisphenol-A typically represents about one half or more of the total capital investment of the system, and known techniques are often very costly and energy intensive.

After the condensation reaction, the resulting mixture is often concentrated, usually by distillation, to remove unreacted Acetone, the Water of reaction, and some Phenol, prior to recovery of the Bisphenol-A product by crystallization. U.S. Pat. No. 5,783,733 describes one prior art method of producing Bisphenol-A wherein Phenol and a ketone are reacted in the presence of an ion exchange resin catalyst to produce a reaction product stream including Bisphenol-A. Prior to crystallization, excess Phenol, Water and Acetone are removed from the product stream. Crystallization, in this case melt crystallization, is used to purify the crude bisphenol. Specifically multiple stage fractional melt crystallization with successive steps of crystallization, partial melting (sweating) and total melting is used. Such Phenol removal and melt crystallization techniques are very costly in terms of capital equipment and energy consumption.

In another well known technique, an adduct of Bisphenol-A and Phenol is first obtained by crystallization, and the adduct is then broken by known methods such as extraction, distillation, dephenolation, steam stripping or prilling, yielding high purity bisphenol A.

This prior art method is described for example in U.S. Statutory Invention Registration US H1943 where the reaction product stream is fed directly to a crystallizer to form a slurry consisting of a liquid phase, and a solid crystal phase of an equal-molar adduct of Bisphenol-A and Phenol. The adduct crystals are separated from the liquid (referred to as mother liquor) and Phenol is removed from the adduct in a series of Phenol removal or dephenolation steps. Finally, multiple stage fractional melt crystallization is preformed to produce the product Bisphenol-A.

The steps to remove Phenol from the adduct are quite costly and add to the complexity of the system. Often, such steps subject the adduct of Bisphenol-A and Phenol to high temperature effects of up to about 250° C., where degradation or undesirable reactions thereof usually occur.

U.S. Pat. No. 4,294,994 describes a method for removal of Phenol from the adduct of Bisphenol-A and Phenol by subjecting the adduct feed at a temperature in the range of about 50° C. to about 150° C., and under spray drying conditions typically at temperatures in the range of about 150° C. to about 250° C. with a small amount of liquid carrier having a boiling point below that of Phenol and recovering the Bisphenol-A product from the released Phenol. The purity of the obtained Bisphenol-A product is up to about 99% by weight; however, this method suffers from a significant disadvantage since the adduct of Bisphenol-A and Phenol experience high temperatures where degradation usually occurs.

Another technique to remove Phenol from the adduct is by distillation as described for example in U.S. Pat. No. 4,798,654. Specifically, the '654 patent describes a process for preparing Bisphenol-A comprising distilling the intermediate adduct of Bisphenol-A and Phenol at a temperature in a range from about 160° C. to about 200° C. in a dephenolization column; recovering Phenol from the top of the distillation column and Bisphenol-A from the bottom of the distillation column; and recycling a part of the bottom liquid to the adduct feed of Bisphenol-A and Phenol. It is said that plugging of the distillation column is prevented and continuous operation for a long period of time such as one year is possible. However, the Phenol content of the Bisphenol-A product taken out of the bottom of the dephenolization column is still up to about 2%.

Many variations to the Bisphenol-A process have been proposed by the prior art. Another example as described in U.S. Pat. No. 4,492,807 provides an improvement in the method of producing an adduct of Bisphenol-A and Phenol which includes the steps of adding up to about 15% by weight of Water and up to 15% by weight of an organic liquid to a mixture comprising impure bisphenol A and Phenol. The organic liquid should not react with Bisphenol-A or Phenol and dissolves a substantial proportion of the impurities or by-products formed in the synthesis of Bisphenol-A. Preferred organic liquids are toluene and Acetone. As described in the '807 patent the impure Bisphenol-A before the adduct crystallization stage may be obtained from the condensation reaction following neutralization if necessary and following removal of volatile components such as unreacted Acetone, Water of reaction and some Phenol. Also, the '807 patent suggests that following the adduct crystallization, the adduct may then be broken by known methods such as extraction, distillation, steam stripping or prilling, yielding high purity bisphenol A. This approach is advantageous in that a pronounced increase in recovery of adduct is obtained without a significant sacrifice in product purity. However, the costly steps of concentration of the impure reaction product mixture before the adduct crystallization stage, and the steps required for recovery of Bisphenol-A from the adduct render this method less desirable.

Another example is described in U.S. Pat. No. 5,723,688, which provides a method of preparing an adduct of bisphenol with a phenolic compound wherein distillation of the reaction product stream prior to crystallization is omitted. Specifically, the '688 patent describes a method of reacting a carbonyl compound with a stoichiometric excess of a phenolic compound in the presence of an acidic cation exchange resin to produce a product mixture containing bisphenol, unreacted phenolic compound, unreacted carbonyl compound and Water. The product mixture is then crystallized to form an adduct of bisphenol with the phenolic compound. Once formed the adduct is preferably washed with a phenolic compound which undergoes costly treatment such that at least a portion of the phenolic compound has been purified by means of an acidic cation exchange resin and a basic cation exchange resin. To recover Bisphenol-A from the adduct, dephenolation steps are employed where the solid adduct is melted and the phenolic compound is recovered by distillation.

While advances have been made in the production of Bisphenol-A, further improvements are needed. The aforementioned prior art methods require costly Phenol removal steps and/or Phenol purification steps, and costly multiple steps to purify the adduct and/or bisphenol. Further, as the purity requirements for Bisphenol-A crystals become more rigorous, the complexity and costs of producing Bisphenol-A increase. Accordingly, it is desirable to provide an improved process for producing Bisphenol-A of high purity.

### SUMMARY OF THE INVENTION

Accordingly, the present invention provides an improved system and method of producing Bisphenol-A.

The system and method of the present invention is described wherein the phase equilibrium of a system comprised of Phenol, Bisphenol-A and a solvent mixture comprising Acetone and Water is selectively controlled such that either crystalline Bisphenol-A, or an adduct of Bisphenol-A and Phenol is crystallized from the product solution. The present invention provides for the recovery of pure Bisphenol-A (BPA) by direct crystallization from solution.

Amont the advantages, this reduces the number of process equipment and steps required to produce Bisphenol-A, thereby reducing capital and energy costs. In addition, this significantly reduces the overall vaporization of Phenol in the process leading to lower energy consumption. It should be understood by those of ordinary skill in the art that the phase equilibrium system may include other components or solutes; however, for purposes of the present invention the system is concerned only with the three primary components—Phenol, Bisphenol-A and a solvent which is comprised of either pure Water, pure Acetone, or preferably, a mixture of Water and Acetone—and thus the phase equilibrium of the system is characterized as a ternary system.

Bisphenol-A is produced from a reaction of Phenol and Acetone, forming a product solution including Phenol, Bisphenol-A, isomers of Bisphenol-A, unreacted reactants and by-products. Acetone and Water are provided in the product solution as the solvent, and the amount of solvent in the product solution is selectively controlled to adjust the composition of the product solution fed to a crystallizer such that an adduct of Bisphenol-A and Phenol is recovered in a first stage crystallization, followed by recovery of substantially pure BPA in a second stage crystallization. Thus, the solvent is used to selectively control the phase behavior of the system.

More specifically, in one embodiment the present invention provides a method of producing Bisphenol-A, comprising the steps of: reacting Phenol and Acetone in the presence of a catalyst to form a product solution including Bisphenol-A, Phenol and where the Bisphenol-A, Phenol and solvent exhibit a phase equilibrium relationship which can be represented by a projection of a polythermal ternary phase diagram. A solvent is provided in the product solution, where the solvent is comprised of Acetone, Water or a mixture of Acetone and Water. The product solution with a selective composition is first fed to a first crystallizer stage operating at a selected temperature such that an adduct of Bisphenol-A and Phenol is crystallized. The formed adduct is then dissolved to obtain a solution with a second selected composition, and fed to a second crystallizer stage operating at a selected temperature such that substantially pure solid Bisphenol-A is formed. If necessary, Bisphenol-A crystals of a higher quality can be obtained via recrystallization of the solid Bisphenol-A. In this instance the second crystallizer stage would contain more than one crystallizer unit providing for the recystallization steps.

Of one advantage, among others, the present invention provides a method for recovering Bisphenol-A by crystallization directly from the solution of the adduct of BPA and Phenol. This eliminates the need for the expensive prior art steps for the complete removal of Phenol from the adduct usually carried out in a dephenolation unit. The system of the present invention only requires a partial removal of the Phenol, which is carried out at a much higher concentration of Phenol, and hence is much cheaper than in the prior art. Further, the present invention provides a method for recovering high quality Bisphenol-A in the crystal form, which eliminates the need for expensive solids formation units such as a prilling tower.

In another aspect of the present invention, a system for producing Bisphenol-A is provided comprising a reactor unit wherein a product stream is produced including at least Bisphenol-A and Phenol. A first stage crystallizer is provided, and the composition of the product stream fed to the first stage crystallizer is selectively adjusted such that an adduct of Bisphenol-A and Phenol is produced upon crystallization in the first stage crystallizer. A dissolution tank is coupled to the first stage crystallizer, where the adduct of Bisphenol-A and Phenol is dissolved; and a second stage crystallizer is provided. The second stage crystallizer receives the product stream from the dissolution stage, and the composition of this product stream is selectively adjusted such that substantially pure Bisphenol-A is produced upon crystallization in the second stage crystallizer.

Further details regarding the various aspects of this invention are provided in the remainder of the disclosure.

### BRIEF DESCRIPTION OF THE DRAWINGS

Other objects and advantages of the present invention become apparent upon reading of the detailed description of the invention provided below and upon reference to the drawings in which:

FIG. 1 is a polythermal phase equilibrium diagram for a binary system of Bisphenol-A and Phenol as found in the prior art.

FIG. 2(a) is the polythermal projection of the phase equilibrium diagram for the ternary system of Bisphenol-A, Phenol and Acetone.

FIG. 2(b) is the polythermal projection of the phase equilibrium diagram for the ternary system of Bisphenol-A, Phenol and Water.

FIG. 3 is the polythermal projection of the phase equilibrium diagram for the ternary system of Bisphenol-A, Phenol and a Solvent which comprises 50% Acetone and 50% Water by weight.

FIGS. 4(a) and 5(a) are polythermal projections of the phase equilibrium diagram for the ternary system of Bisphenol-A, Phenol and Solvent showing the regions of composition of the crystallizer feed, which are preferable from the point of view of process operation according to one embodiment of the present invention.

FIGS. 4(b) and 5(b) are polythermal projections of the phase equilibrium diagram for the ternary system of Bisphenol-A, Phenol and Solvent showing the regions of composition inside the crystallizer, which are preferable from the point of view of process operation according to one embodiment of the present invention.

FIG. 6 is a schematic diagram illustrating a process configuration for producing Bisphenol-A according to one embodiment of the system of the present invention.

FIG. 7 is the polythermal projection of the phase equilibrium diagram for the ternary system of Bisphenol-A, Phenol and Solvent showing the composition of the key process streams for the system described in FIG. 6.

FIG. 8 is a schematic illustration showing the details of the Solvent recovery system which may be employed in the system described in FIG. 6.

### DETAILED DESCRIPTION OF THE INVENTION

Background of Phase Diagrams and Projections

The inventors have discovered a system and method of producing bisphenol wherein the phase equilibrium behavior of the product stream fed to a crystallizer stage (sometimes also referred to as the feed solution to the crystallizer stage) is selectively controlled to provide selected results for process operation. The invention provides for manipulation and selective control of the phase equilibrium of Bisphenol-A, Phenol and a solvent. It should be understood by those of ordinary skill in the art that the system may include other components or solutes such as impurities, unreacted reactants, isomers and the like; however, for purposes of the present invention we are concerned only with the primary components in the solution—Phenol, Bisphenol-A and solvent—where the solvent is a pseudo-component representing either pure Water, pure Acetone, or preferably, a mixture of Water and Acetone, and thus the system is characterized as a ternary phase equilibrium system.

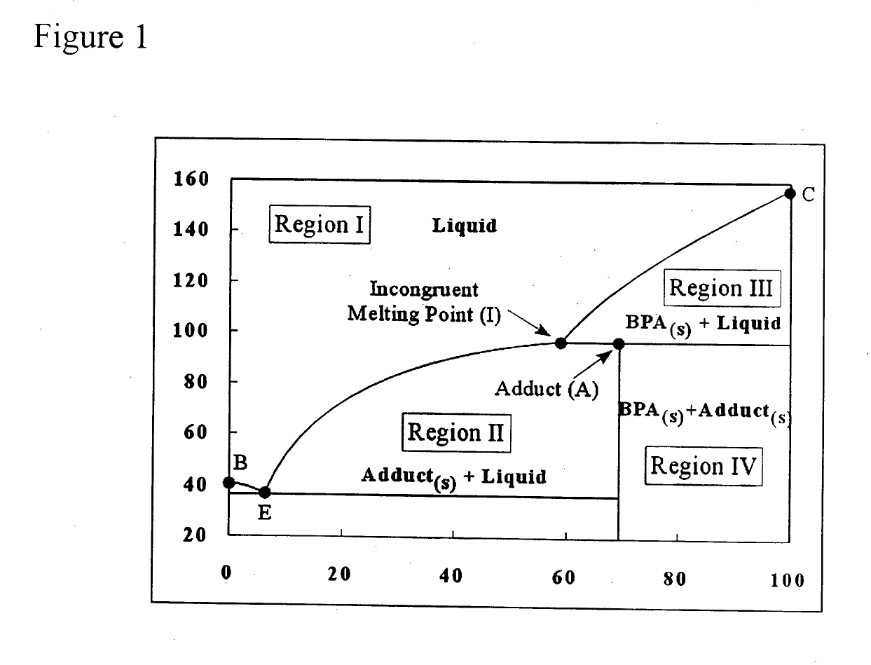

In the prior art, the phase equilibrium of the Bisphenol system for crystallization is typically considered as a binary system comprised of Bisphenol-A and Phenol as shown in FIG. 1. Since pressure has little effect on the equilibria between solids and liquids, the phase changes for a binary system can be represented on a temperature-concentration diagram. A plurality of phase regions are shown. In Region I, bounded by the liquidus transition line BEIC, both components (i.e. Bisphenol-A and Phenol) are in the liquid phase. A eutectic between Phenol and the adduct of Phenol and Bisphenol-A exists at point E. An incongruent melting eutectic between Bisphenol-A and the adduct exists at point I. The incongruent melting point (I) is the point where the adduct melts, and the liquid composition and the solid composition are not the same. When the adduct of Phenol and Bisphenol-A melts, the liquid composition is at point I while some solid Bisphenol-A is formed as well. This is why the location of the adduct (point A) is between point I and pure Bisphenol-A. In Region II the solid adduct exists in equilibrium with liquid. The solid adduct is comprised of an equal-molar composition of Bisphenol-A and Phenol. In Region III solid Bisphenol-A exists in equilibrium with liquid. In Region IV both solid adduct and solid Bisphenol-A phases exist. Such a phase diagram which illustrates the relation between the temperature and composition is referred to as a polythermal phase diagram.

The approach of the prior art for the separation of Phenol from Bisphenol-A is to describe a system with only two components and this can be described by use of the binary phase diagram as illustrated in FIG. 1. However for systems with more than two components as taught by the present invention, the polythermal phase diagram is a figure in multi-dimensional space. For example, the isobaric phase diagram of the ternary system of Phenol, Bisphenol-A and solvent represents an image in 3-dimensional space. The composition coordinates can be plotted in weight fractions on a triangular grid in the X-Y plane, and the temperature can be plotted on an additional vertical axis (Z axis). This leads to the polythermal phase diagram in the shape of a 3-dimensional (3D) triangular prism. However, it is not convenient to work with multi-dimensional phase diagrams. Fortunately, for this three component system, much of the important information, particularly the crystallization boundaries, can be represented on a 2-dimensional (2D) projection onto a triangular base. This is obtained by projecting the phase diagram along the temperature axis (Z axis), onto the base of the prism (X-Y plane), and is referred to as the projection of a polythermal phase equilibrium diagram.

It is a common practice at the conceptual design stage to reduce a 3D diagram to a 2D diagram as described above, for ease of visualization and for process design purposes. The projection of the polythermal diagram can be employed as a useful tool for process feasibility study because it provides the region for product recovery.

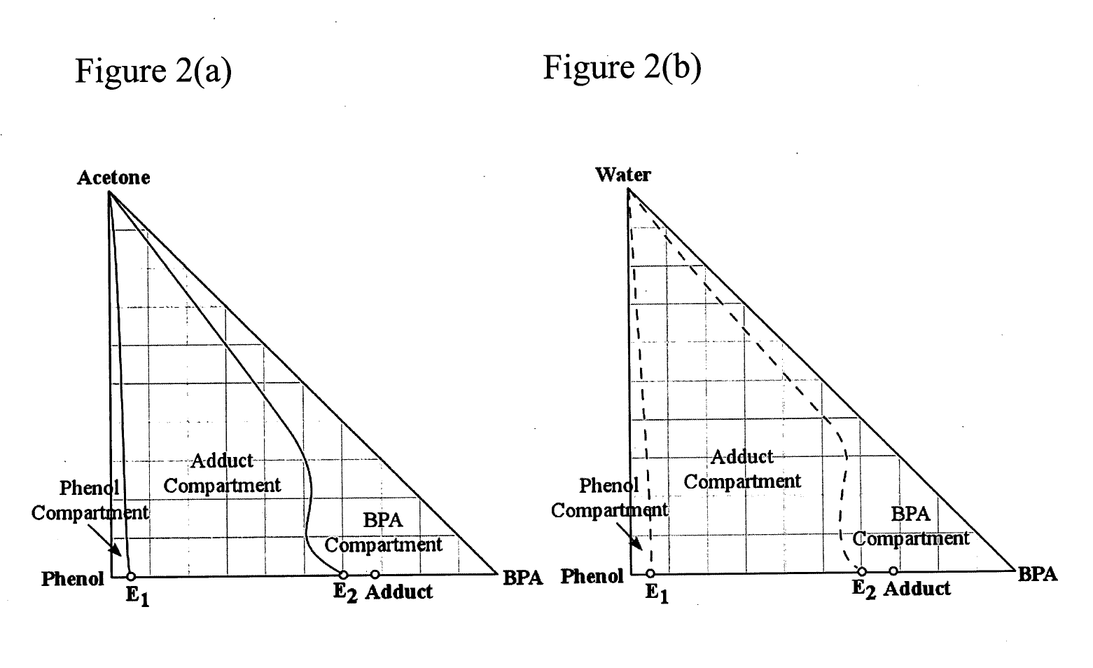

FIG. 2(a) illustrates the projection of the polythermal phase diagram of a ternary system comprising of Phenol, Bisphenol-A, and Acetone. Please note that the ternary phase equilibrium diagram is represented herein as a right-angled triangle for clarity, as opposed to the alternative equilateral triangular diagram form. There are two important regions or compartments in the phase diagram for production of Bisphenol-A. One is the Bisphenol-A compartment and the other is the adduct compartment. These are identified in the illustration in FIG. 2(a). The Phenol compartment occupies only a small portion of the phase diagram, and is not important from the consideration of recovering solid Bisphenol-A. Also, a compartment where Acetone can be recovered as a solid exists near the solvent vertex. However, for Acetone and other low-boiling solvents this compartment is so small and so far away from the desired region of operation, that it can be ignored and hence has not been shown in FIG. 2(a).

When the feed composition to the crystallizer is located inside the Bisphenol-A compartment, substantially pure solid Bisphenol-A can be recovered by various means, such as cooling the mixture to an appropriate temperature, or by evaporating the solvent. When the feed composition to the crystallizer is located in the adduct compartment, solid adduct can be recovered. Thus, the projection of the polythermal phase diagram clearly delineates the regions in the composition space where a feed stream or feed solution to a crystallizer can yield solid adduct or solid Bisphenol-A. FIG. 2(b) shows the projection of a polythermal diagram for a ternary system comprising of Phenol, Bisphenol-A, and Water. Note that as before, four phase regions are established in this phase diagram—a Bisphenol-A compartment, an adduct compartment, a Phenol compartment, and a Solvent (in this case Water) compartment (not shown). However, the shape and location of the phase boundaries for Acetone and Water solvents are somewhat different. This is because the phase behavior of the system results from the mutual interaction of all the components present in the system, and different solvents produce a different effect on the phase behavior of the overall system.

The actual experimental data for the Phenol-BPA-Water and Phenol-BPA-Acetone system may be determined by those of ordinary skill in the art without undue experimentation, and is discussed in further detail in a separate section later.

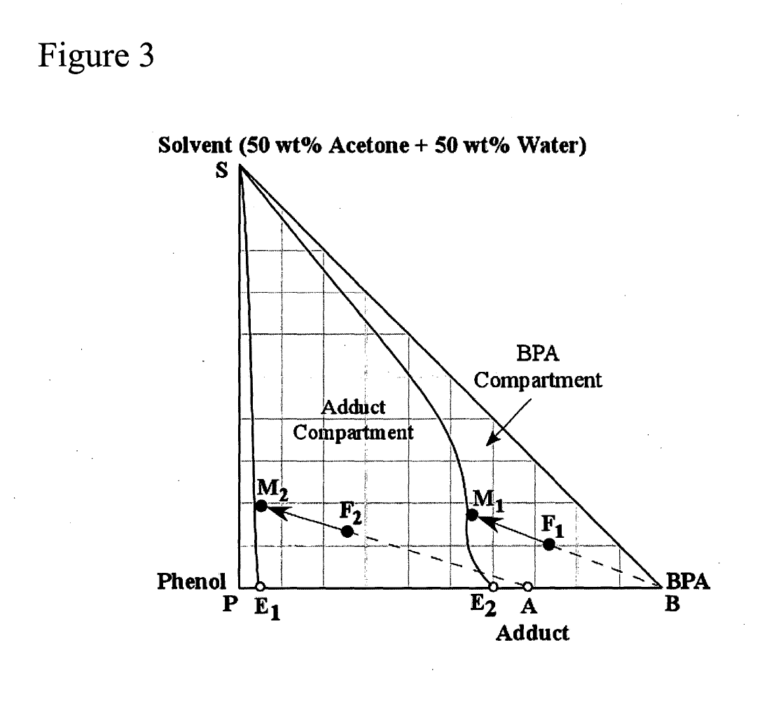

In addition to identifying the regions of product recovery, the projection of the polythermal phase diagram can also be used to identify the maximum recovery of the solid from the crystallizer. Referring to FIG. 3 the projection of a polythermal phase diagram is illustrated for a ternary system of Bisphenol-A, Phenol, and a Solvent which is comprised of a mixture of 50% Acetone and 50% Water on a weight basis. Consider a feed solution to the crystallizer located at point F₁ in the BPA compartment, as shown in FIG. 3. During crystallization, crystals of solid Bisphenol-A are recovered from the crystallizer, while the composition of the mother liquor is given by a point on the line indicated by the arrow F₁M₁. The recovery of Bisphenol-A can be defined as the ratio of the mass of pure Bisphenol-A solid recovered to the mass of the feed. In FIG. 3, the maximum recovery of Bisphenol-A is given by the ratio of F₁M₁ to BM₁, because both Bisphenol-A and adduct solid would co-exist once the composition of the mother liquor reaches the phase boundary SE₂. A feed with composition represented by point F₂ lies in the adduct compartment. Upon crystallization, such a feed will yield solid adduct while the composition of the mother liquor is given by a point on the line indicated by the arrow F₂M₂. The maximum recovery of the adduct is given by the ratio of F₂M₂ to AM₂, because both adduct and solid Phenol would co-exist once the composition of the mother liquor reaches the phase boundary SE₁.

Feasible movement for the system within the composition space can be provided by varying or changing of one or more of the following three parameters: temperature (typically cooling), adding of an “anti solvent,” meaning a component that has the opposite effect of the solvent, or evaporation of the solvent.

When considering the process design, it is important to control the composition, and hence the location on the phase diagram, of two points, namely the feed to the crystallizer, and the outlet from the crystallizer (the outlet from the crystallizer is commonly referred to as the mother liquor). In order to only crystallize the desired product, both these two points must lie within the desired product's compartment in the phase diagram. The two points will be collinear with a third point, which is the composition of the solid being crystallized. Also, to maximize the per pass recovery of the desired solid product from the crystallizer, the two points representing the compositions of the crystallizer feed and the crystallizer outlet (the mother liquor) should have a large separation in their location. Thus, a good crystallizer design will try to control the two points within the same compartment while having a maximum distance between the two.

Method of the Present Invention

The present invention is now described in more detail. In contrast to the prior art, the inventors have discovered a system and method of producing Bisphenol-A wherein three components are employed—Bisphenol-A, Phenol and a solvent comprised of either pure Acetone, pure Water, or a mixture of Acetone and Water-and the ternary phase conditions are selectively controlled. This is a very powerful tool and allows one to manipulate the phase equilibrium conditions by varying process operating conditions such as, but not limited to, the type of solvent(s); concentration ratio of solvent components employed; mass concentration of any one of, or combination of, Phenol, Bisphenol-A and the solvent fed to the crystallizer; feed rate of any one of, or combination of, the reactants fed to the condensation reactor; or recycled feed rates of any of the relevant constituents.

In one embodiment, the present invention provides a system wherein the phase equilibrium behavior of a feed solution to a crystallizer is selectively controlled and adjusted to produce a solid adduct of Bisphenol-A and Phenol in a first crystallization stage and then substantially pure Bisphenol-A crystals are crystallized directly in a second crystallization stage. This provides a powerful tool, whereby certain operating conditions or variables of the Bisphenol-A process can be selectively adjusted to generate desired phase equilibrium behavior.

More specifically, in one example of this embodiment, a method of producing first an adduct of Bisphenol-A and Phenol from a product solution in a first crystallization stage is provided, wherein the product solution is produced by a condensation reaction of Phenol and Acetone, and is comprised of at least Phenol, Bisphenol-A, isomers of Bisphenol-A, and un-reacted reactants. A solvent preferably comprising either pure Water, pure Acetone, or a mixture of Water and Acetone is provided in the product solution. Those of ordinary skill in the art will recognize that other solvent components may be used. In one embodiment the solvent is comprised of a mixture of Acetone and Water at a composition in the range of 99 wt % Acetone and 1 wt. % Water to 1 wt. % Acetone and 99 wt. % Water. In another embodiment the solvent is comprised of a mixture of Acetone an Water at a composition in the range of 40 wt. % Acetone to 60 wt. % Water to 60 wt. % Acetone and 40 wt. % Water. In a further embodiment the solvent is comprised of a mixture of about 50 wt. % Acetone and 50 wt. % water. Water and Acetone are conveniently employed as they are already present in the product solution. The solvent may be added to the product solution after the reaction. Alternatively, at least a portion of the solvent may already be present in the product solution following the reaction. The product solution is fed to a first stage crystallizer and the phase equilibrium behavior of this feed stream to the crystallizer is selectively controlled by providing the product solution having a certain composition, such that an adduct of Bisphenol-A and Phenol is crystallized from the product solution. The phase equilibrium behavior is selectively controlled most usually by controlling the concentration and composition of the solvent in the product solution. However, the concentration of the other components in the product solution may also be controlled.

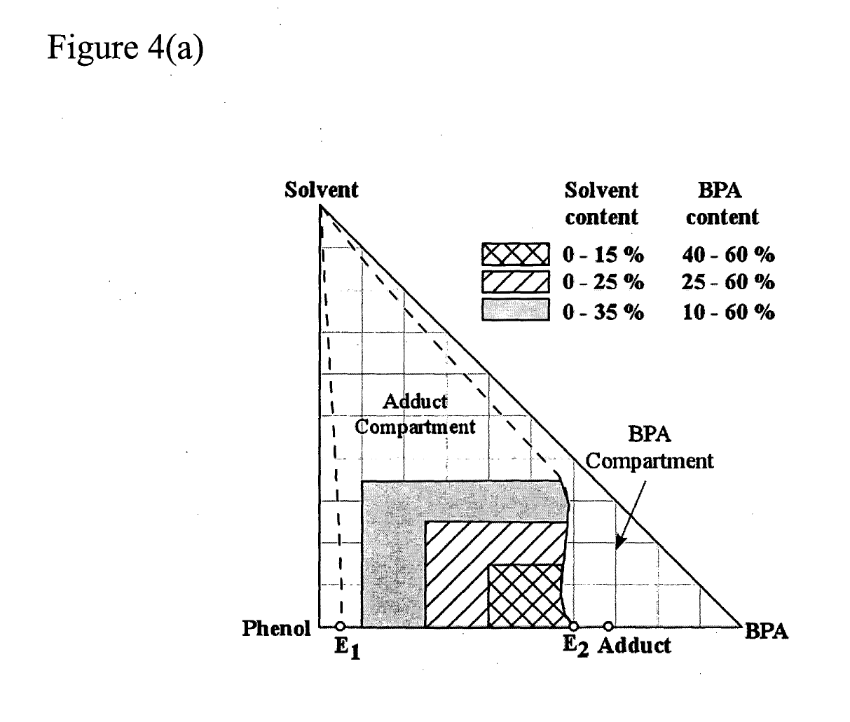

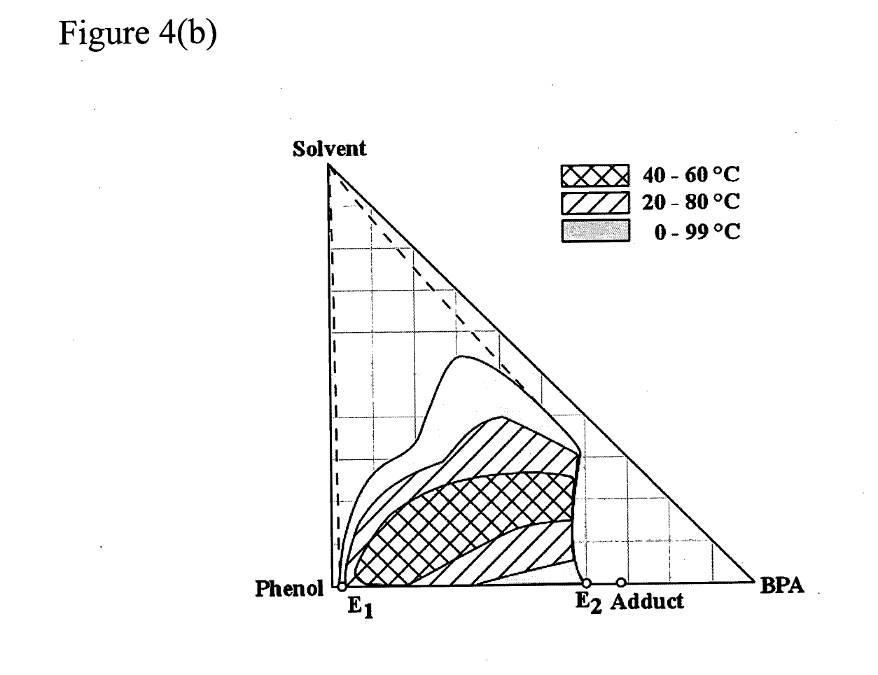

Referring specifically to FIGS. 4(a) and 4(b), the projection of a polythermal phase diagram of the ternary system of Phenol, Bisphenol-A and Solvent is shown. FIGS. 4(a) and 4(b) are shown to illustrate the first stage crystallization. The components exhibit a phase behavior which establishes at least two regions or compartments in the phase diagram, an adduct phase compartment and a pure solid Bisphenol-A phase compartment. A solvent compartment is also present, but is not discussed in detail since it is not of significant interest to the teaching of the present invention. The location of the boundary of the compartment, and thus the size of the adduct compartment and the Bisphenol-A compartments, are selectively controlled or manipulated by adjusting the composition of the solvent, or in the case where the solvent is a mixture, such as of Acetone and Water, by adjusting the concentration ratio of the two solvent components. In this first crystallization step the composition of the feed stream, and hence the location of the feed point on the phase diagram is adjusted so that the feed composition lies in the adduct region. Preferably, the feed stream composition is such that it lies in the shaded area shown in FIG. 4(a). In this example the concentration of solvent in the feed is in the range of about 0 to 35 wt % and the concentration of Bisphenol-A in the feed is in the range of about 10 to 60 wt %. More preferably, the concentrations of solvent and Bisphenol-A in the feed are in the range of 0 to 25 wt % and 25 to 60 wt %, respectively. Most preferably the concentrations of solvent and Bisphenol-A in the feed are in the range of about 0 to 15 wt % and 40 to 60 wt %, respectively.

FIG. 4(b) shows a way of depicting the preferable area of the composition of the mother liquor inside the crystallizer, based on the temperature of operation of the crystallizer. In this example as shown in FIG. 4(b), the region of the Phenol-BPA-Solvent composition space is depicted, where the adduct crystallizer operates in a temperature range between approximately 0 and 99° C., more preferably in the range of 20 to 80° C., with a temperature range of approximately 40 to 60° C. being most preferred.

Those of ordinary skill in the art will recognize that the size of the compartments in FIGS. 4(a) and 4(b) may move somewhat if impurities or other components are present in the product solution.

Once the adduct is formed in the first crystallization step, the adduct is dissolved, again forming a product solution comprised of at least Phenol and Bisphenol-A, where the product solution is produced by dissolving the adduct of Bisphenol-A and Phenol.. A solvent comprising for example either pure Water, pure Acetone, or a mixture of Water and Acetone is provided in the product solution. The solvent may be added to the product solution after the adduct crystallization. Alternatively, at least a portion of the solvent may already be present in the product solution. The product solution is fed to a second stage crystallizer and the phase equilibrium behavior of this feed stream to the second stage crystallizer is selectively controlled by providing the product solution having a second selective composition, such that substantially pure Bisphenol-A is crystallized from the product solution. The concentration of the solvent and/or the concentration of other components in the product solution may be controlled to achieve the desired phase behavior.

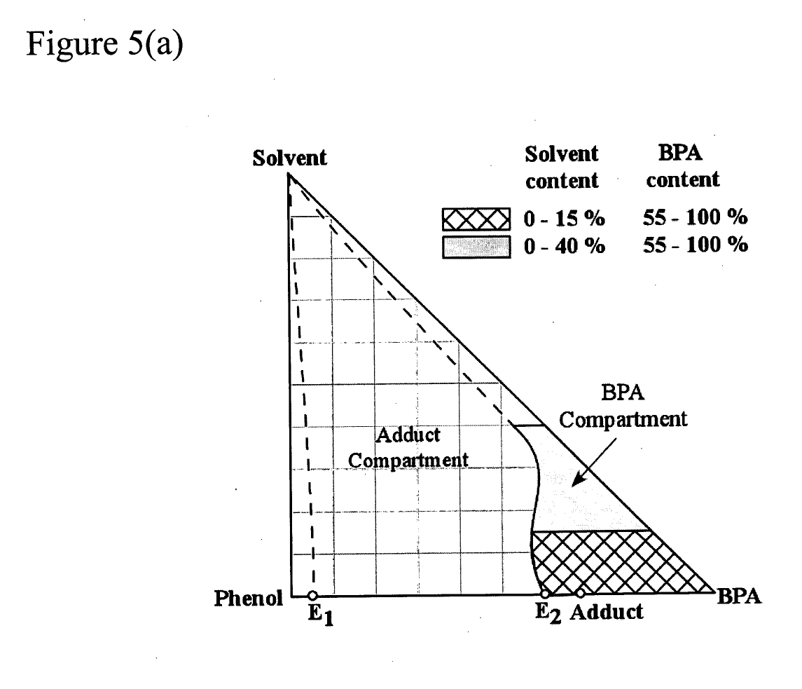

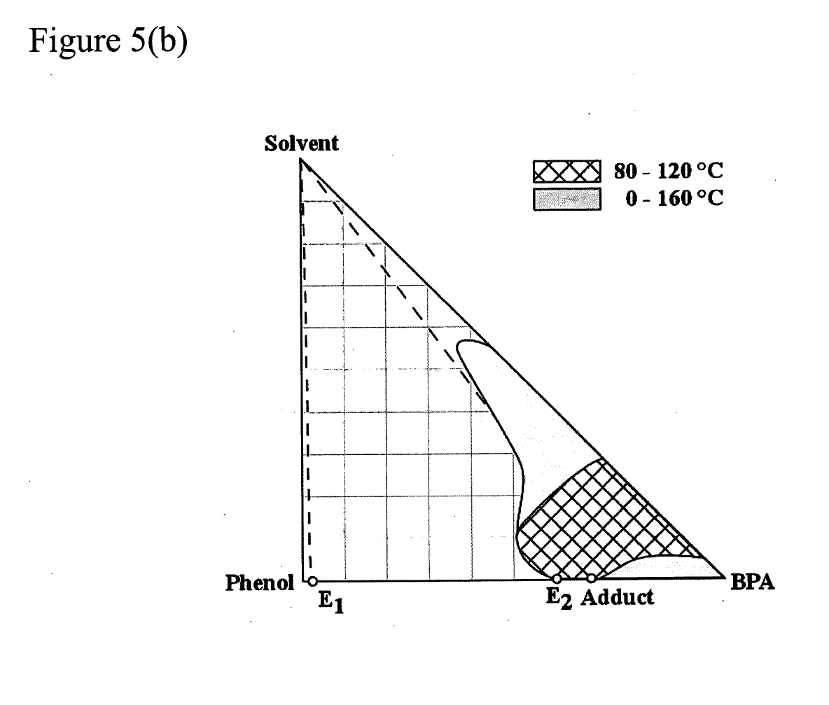

To recover substantially pure Bisphenol-A, we specifically refer to FIGS. 5(a) and 5(b), which show the projection of a polythermal phase diagram of the ternary system of Phenol, Bisphenol-A and Solvent. FIG. 5(a) and 5(b) are shown to illustrate the second stage crystallization. The phase boundaries are established by providing a solvent as described earlier. The composition of the feed solution to the second stage crystallization, and hence the location of the feed point on the phase diagram is adjusted so that the feed stream composition lies in the Bisphenol-A compartment. Preferably, the feed stream composition is such that it lies in the shaded area shown in FIG. 5(a). In this example the concentration of solvent in the feed is in the range of about 0 to 40 wt % and of the concentration of Bisphenol-A in the feed is in the range of about 55 to 100 wt/%. More preferably, the concentrations of solvent and Bisphenol-A in the feed are in the range of 0 to 15% and 55 to 100%, respectively.

Again, FIG. 5(b) shows a way of depicting the preferable area of the composition of the mother liquor inside the crystallizer, based on the temperature of operation of the crystallizer. Preferably the crystallizer mother liquor composition is such that it lies in the shaded area shown in FIG. 5(b) when the crystallizer operates at a temperature in the range of approximately 0 to 160° C., and most preferably in the range of about 80° C. to 120° C.

It is important to note that although the feed solution to the first or second crystallizer stages is comprised of a multitude of components, including unreacted reactants and byproducts of the reaction, for process synthesis purposes it is sufficient to consider only the principal components. The principal components are Phenol, Bisphenol-A and the solvent (a pseudo-component generically referred to as the solvent). As discussed above, the solvent component may be a single component such as pure Water or pure Acetone. In a preferred embodiment the solvent is comprised of a mixture of Acetone and Water in varying proportions. The solvent components may already be present in the product solution available from the reaction step, such as unreacted Acetone and the Water formed in the reaction, or may be introduced into the system before the crystallization step. The system of Phenol, Bisphenol-A and the solvent is treated as a ternary phase equilibrium system whose phase behavior will depend on the exact nature of the solvent.

Calculating Phase Diagrams

Given the teaching of the present invention, ternary phase equilibrium diagrams for different solvent compositions and/or concentration ratios may be created by those of ordinary skill in the art without undue experimentation. For example, computer simulation programs and thermodynamic databases may be used to develop the approximate location of the boundary of the bisphenol- and adduct compartments for different pure solvents and/or solvent mixtures with different concentration ratios. The boundary between the adduct compartment and the Bisphenol-A compartment is the trajectory of incongruent melting points. The incongruent melting point is the location where both Bisphenol-A and adduct solid coexist. The maximum recovery of Bisphenol-A is the maximum amount of Bisphenol-A solid that can be recovered before adduct solid appears (or coexists with the Bisphenol-A solid). It should be noted that the polythermal phase diagrams shown herein are not to scale but are presented here for schematic illustration purposes.

More specifically, the establishment of a thermodynamic model for the multi-component solid liquid equilibrium uses both theoretical calculation and experimental data for the model verification. Those of ordinary skill in the art can develop such thermodynamic models using known principles and tools.

The physical properties of the each component in the feed solution to the crystallizer such as the heat of fusion, melting point, and heat capacity are used to establish the thermodynamic model which describes the equilibrium relationship between the components as a function of temperature. The theory for the activity coefficient is well developed, and a computer software program providing numerical methods for estimating the equilibrium relationships between the components is typically used. The binary interaction parameters among the components in the solution are either estimated or determined from experimental data. Examples of suitable commercially available software programs for thermodynamic model establishment include PropertiesPlus from Aspen Technology Inc., HYPROP III from Hyprotech, and ProPred from CAPEC at the Technical University of Denmark. The thermodynamic model is for the calculation of the concentration of the component when it is saturated and is a function of temperature and concentration. Since the effect of pressure on the saturation is not very significant in the pressure range considered, the pressure effect is not taken into account in this thermodynamic model; however, the analysis may be extended to consider the pressure effect if desired.

The region of the Bisphenol-A compartment can be determined if the trajectory of the incongruent melting points can be determined. Since the incongruent melting point occurs when both adduct and Bisphenol-A are saturated, this trajectory can be calculated from the thermodynamic model by those of ordinary skill in the art. The regions for the Phenol compartment can be calculated once the trajectory of the eutectic point between Phenol and adduct is determined. This trajectory can be also calculated by those of ordinary skill in the art from the thermodynamic model since the eutectic points occur when both Phenol and adduct are saturated. The region between the Phenol compartment and the Bisphenol-A compartment in the concentration diagram is the adduct compartment. The temperature along the trajectory of incongruent melting points and the trajectory of the eutectic points is also be determined from the thermodynamic model by known techniques. Further detailed description regarding the development of the phase diagrams may be found in pending U.S. patent application Ser. No. 10/099,227 (Attorney Docket no. A-71155/MSS) filed on Mar. 13, 2002, the entire disclosure of which is expressly incorporated herein by reference.

System (Process Configuration) of the Present Invention

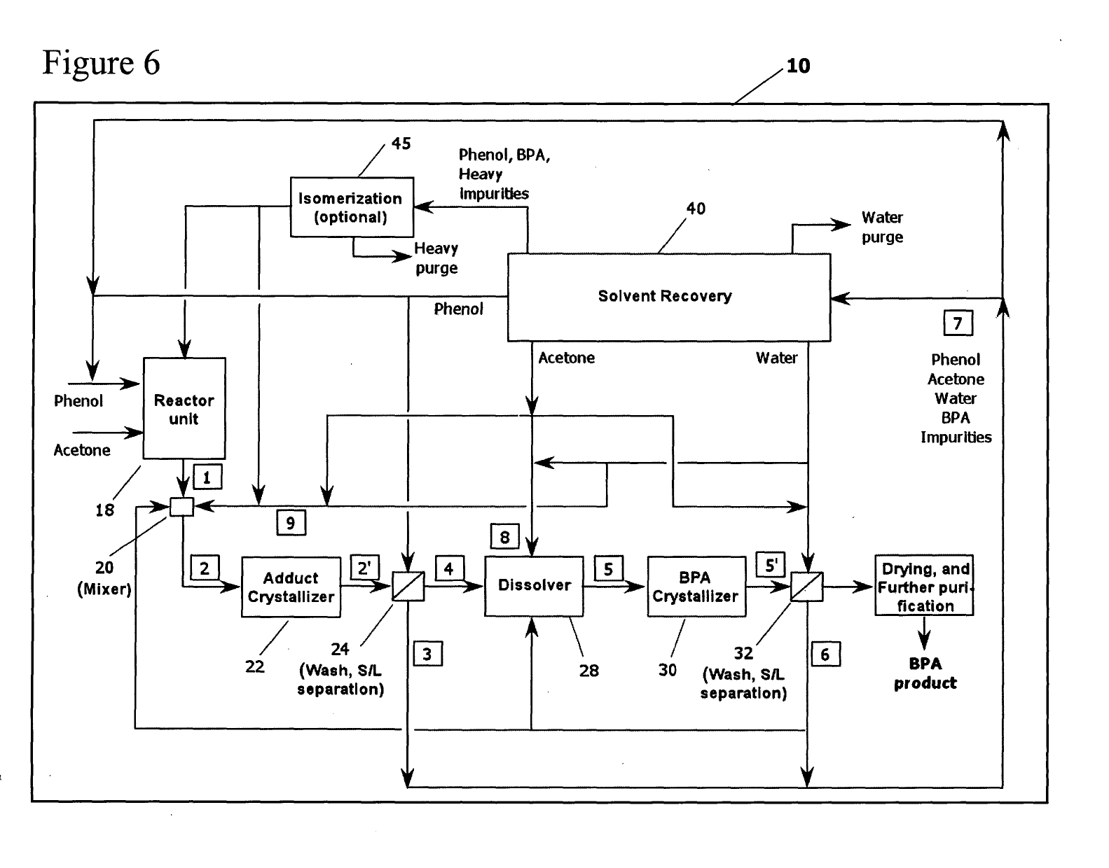

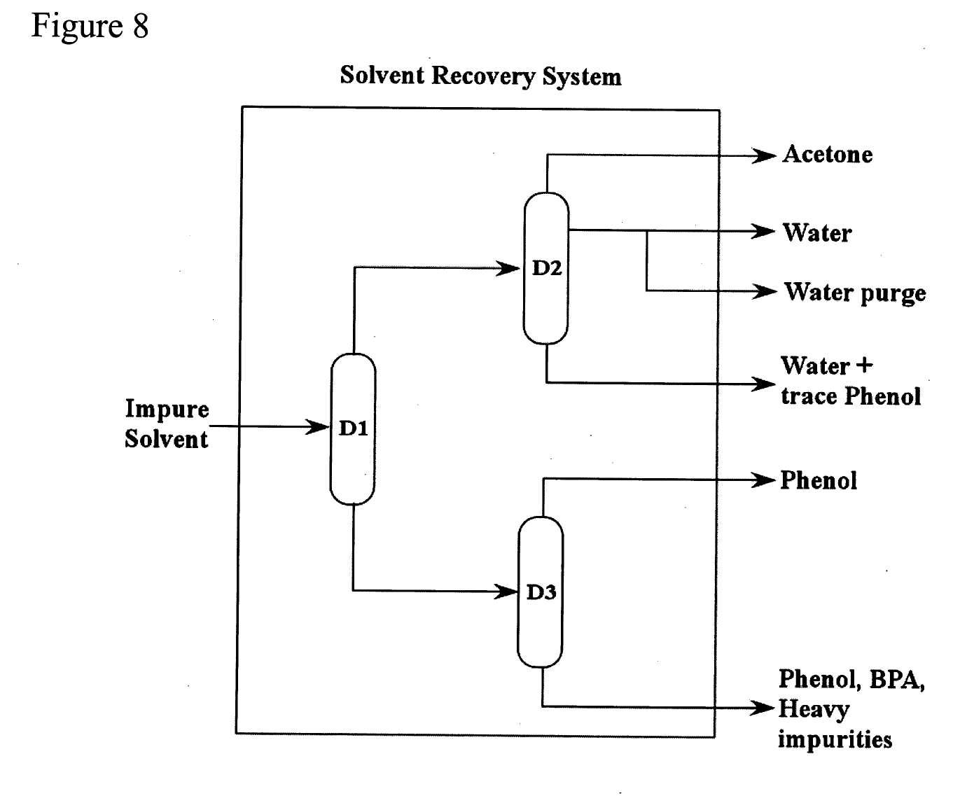

Of particular advantage, the present invention provides a system for producing Bisphenol-A, said system employing a two stage crystallization. One embodiment of the system of the present invention is illustrated in FIG. 6. In general, the system 10 is comprised of a reactor unit 18, first crystallizer stage 22 (also referred to herein as an adduct crystallizer), dissolver tank 28 and a second crystallizer stage 30 (also referred to herein as a BPA crystallizer). Downstream of the reactor unit 18, a mixer/separator 20 may be employed to adjust the composition of the solvent in the product solution prior to the first crystallizer stage 22. Downstream of the first and second crystallizer stages 22, 30, a first solid/liquid (S/L) separator 24 and then a second S/L separator 32, respectively, may be employed. The Bisphenol-A crystals produced from the second crystallizer 30 may be further processed by drying, followed by further purification methods such as recrystallization and the like, although not shown. A solvent recovery unit 40 may be employed to recover and/or recycle solvent and Phenol to the system 10. FIG. 8 shows one illustration of a solvent recovery system which may be used to recover and recycle the solvent and Phenol in System 10.

To form Bisphenol-A, Phenol (preferable purified and preferably in stoichiometric excess) and Acetone are conveyed to the reactor unit 18 where a condensation reaction is carried out. Reactor unit 18 is preferably a fixed bed reactor and the reactants are passed through the fixed bed having an acid catalyst to produce a product solution in the liquid phase. The product solution includes Bisphenol-A, unreacted reactants such as excess Phenol, by-products of the reaction (such as Water) and impurities such as isomers, analogs and homologs. The condensation reaction may be carried out at a temperature in the range of about 45° C. to 120° C., and more preferably in the range of about 50° C. to 100° C. with about 75° C. being most preferred. The reaction pressure is in the range of about 1 to 8 bar, more preferably about 1 to 6 bar, with a reaction pressure of about 4.4 bar being most preferred.

The system 10 provides two crystallizers stages, namely, the first or adduct crystallizer stage 22 to form an adduct of Bisphenol-A and Phenol, and the second or BPA crystallizer stage 30 to form Bisphenol-A crystals. As described above, according to the present invention, a solvent (in one example comprised of pure Acetone, pure Water or mixtures thereof in various concentration ratios) is employed to establish the phase equilibrium compartments for solid adduct and Bisphenol-A in the respective crystallizer stages.

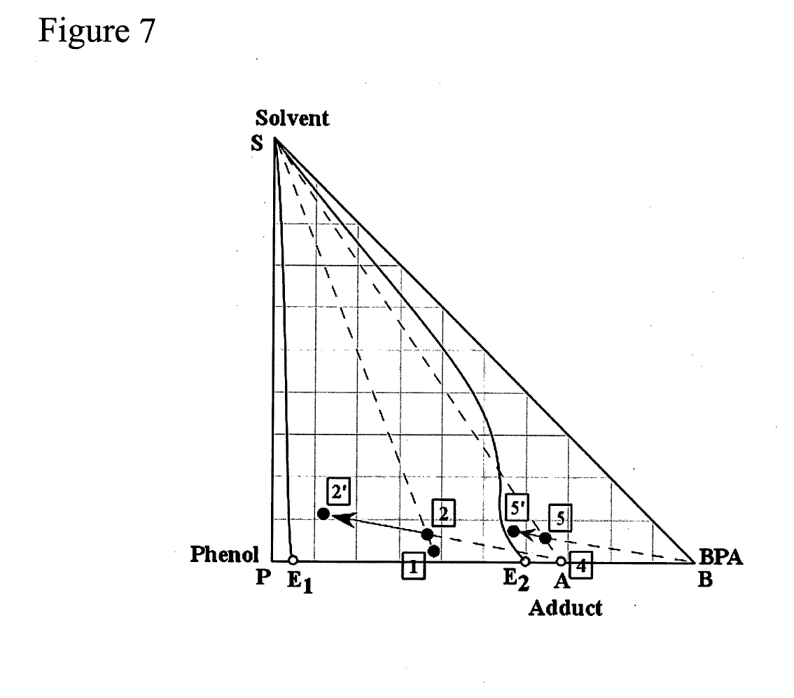

The product solution following the reaction is identified as Stream number 1 on FIG. 6. The composition of this stream on an impurity free basis can be plotted on a polythermal phase diagram of the ternary system comprised of Bisphenol-A, Phenol, and Solvent, as shown in FIG. 7. The composition of Stream 1 is identified by point 1 in FIG. 7. The composition of the feed solution fed to the adduct crystallizer 22 and the Bisphenol-A crystallizer 30 is adjusted by employing a suitable solvent such that the composition of the feed stream falls within the adduct phase compartment (shown on FIG. 7) in the adduct crystallizer 22 and within the Bisphenol-A phase compartment (shown on FIG. 7) in the Bisphenol-A crystallizer 30. The feed streams to the adduct crystallizer 22 and the Bisphenol-A crystallizer 30 are shown in FIG. 6 as streams 2 and 5, respectively. The corresponding locations of these feed compositions in the crystallizers are shown as points 2 and 5, respectively, on the projection of the polythermal phase diagram shown in FIG. 7.

Following the reaction, the product solution is fed to the first, or adduct crystallizer stage 22. According to one embodiment of the present invention the composition of the product solution is adjusted in mixer/separator 20 prior to entering the adduct crystallizer so as to establish a feed stream having a feed composition at a location within the adduct phase region such that the adduct is formed upon crystallization. In one example the feed composition to the adduct crystallizer stage is in the range from about 25 to 60 wt % BPA and from about 0 to 25 wt % solvent when the adduct crystallizer stage is operated at a temperature in the range of approximately 40° C. to 60° C. In this exemplary embodiment the composition of the feed stream is adjusted from a composition corresponding to point 1 on FIG. 7 to a composition corresponding to point 2 by the addition of various components—in this case by recycle stream 9 comprised of a solvent of Acetone and Water having a composition corresponding to point S. In the exemplary embodiment the solvent is supplied from the solvent recovery unit 40, and the composition of the solvent in Stream 9 is adjusted by mixing appropriate amounts of Acetone and Water recovered from the solvent recovery unit 40. While one example is shown here, the solvent and/or other components may be added prior to each of the crystallizer stages 22 and 30 by any suitable means; and is preferably added as one or more recycle streams from the solvent recovery unit 40 and/or from any one or both of the S/L separators 24 and 32. For example, a part of the filtrate (Stream 6) from the second stage washing unit/ S/L separator 32 may be recycled to the first stage crystallizer 22. Also, a part of the Phenol recycle stream (Stream 10), or a part of the Phenolic stream containing BPA and other products (Stream 12) may be recycled to the Adduct crystallizer 22. Similarly, the composition of the feed to the adduct crystallizer can be adjusted by adding one or more recycle streams from the processing units downstream of the second stage washing/solid-liquid separator unit 32.

Known process control systems, such as on-line analysis with feedback control, may be used to selectively adjust the composition of the product solution at the feed to the crystallizer stages. Also in some cases, solvent in the desired concentration may already be present in the product solution following the reaction and/or the first crystallizer 22, and thus physical addition of the solvent may not always be necessary.

Having achieved the desired feed composition for adduct crystallization, the solution is fed to the adduct crystallizer stage 22 whereupon a first crystallization step is conducted to crystallize solid adduct of Phenol and Bisphenol-A. The adduct crystallizer stage 22 may be comprised of one or more crystallization units. Crystallizers are known in the art, and typically provide crystallization by indirect or external cooling such as with heat exchanges or circulating cooling medium. Crystallization may also be provided by pressure reduction, or by a combination of external heating and pressure reduction. In one example, the solution is cooled in the adduct crystallizer 22 to a temperature in the range of 0° C. to 99° C., more preferably to a temperature in the range of about 20° C. to 80° C., and most preferably in the range of 40° C. to 60° C. The operating pressure range depends on the type of solvent used and the crystallizer temperature, and the pressure should be selected such that the vapor fraction in the crystallizer is low or minimized. The adduct crystallizer is typically operated at a pressure in the range of about 0.1 bar to 6 bar. In an exemplary embodiment, where Acetone is employed as the solvent the pressure in the adduct crystallizer 22 is in the range of about 0.5 bar to 5 bar, with about 3 bar being most preferred to maximize the liquid fraction in the adduct crystallizer 22.

As cooling takes place, solid adduct of Phenol and Bisphenol-A form in the adduct crystallizer 22. The residence time in the adduct crystallizer 22 is in the range of about 1 to 10 hours ideally, more preferably in the range of about 2 to 5 hours. It should be understood that the optimum residence time is a function of the crystal growth rate in a given solvent, and thus the optimum residence times will vary depending on the type of solvent employed.

The solid adduct is separated from the remaining solution ( referred to as mother liquor or M/L) in the first S/L separator 24. Any suitable type of S/L separator as known in the art may be used such as centrifuge or filtration device, and the like. Upon separation the adduct will have a composition corresponding to point 4 in FIG. 7. The mother liquor corresponding to stream 3 in FIG. 6, is conveyed to the solvent recovery unit 40 where solvent and Phenol are separated and recycled to the reactor, and/or may be added independently to mixer/separator 20 and dissolution tank 28 to adjust the feed composition as desired prior to the crystallization steps. The solvent recovery unit 40 may employ any suitable separation means such as distillation and the like. The Phenol and solvent components may also be used to wash the adduct and the Bisphenol-A crystals in order to remove the residual mother liquor. The adduct can be washed with purified Phenol in S/L separator 24.

Following separation, the adduct is dissolved in dissolution tank 28 at an elevated temperature. The elevated temperature is selected to be above the dissolution temperature of the adduct, and is dependent upon the concentration of the solvent. In the preferred embodiment, solvent is further added to the dissolution tank 28 to assist with dissolving the adduct. The solvent composition is chosen to selectively establish the Bisphenol-A and adduct compartments in the phase equilibrium behaviors desired, and the composition of the product solution is adjusted to place the composition of the feed stream to the Bisphenol-A crystallizer stage 30 within the desired Bisphenol-A phase compartment. In this example, the composition of the product solution is adjusted by the addition of a recycle stream 9 from the solvent recovery unit 40 which has a composition corresponding to point S on FIG. 7. Preferably, solvent is added in the dissolution tank; however, the solvent may be added at any other location prior to the Bisphenol-A crystallizer stage 30. Apart from the solvent other components or streams may be added to the dissolver unit 28, or prior second crystallization stage 30 by any suitable means, in order to achieve the desired composition of the feed stream to the BPA crystallization stage 30. For example, a part of the filtrate (Stream 6) from the second stage washing unit/S/L separator 32 may be recycled to the dissolver unit 28. In one example embodiment, the feed composition to the BPA crystallizer stage is in the range from about 55 to 100 wt % BPA and from about 0 to 15 wt % solvent.

The dissolved adduct stream, having a composition corresponding to point 5 on FIG. 7, is sent to the Bisphenol-A crystallizer stage 30 to crystallize substantially pure Bisphenol-A. The Bisphenol-A crystallizer stage 30 may be comprised of one or more crystallizer units. The solution is typically cooled in the Bisphenol-A crystallizer 30 to a temperature in the range of about 0° C. to 160° C., more preferably to a temperature in the range of about 80° C. to 120° C. The Bisphenol-A crystallizer 30 is typically operated at a pressure in the range of about 0.1 bar to 6 bar. Again, any suitable crystallizer may be used, and of particular advantage, the size of the Bisphenol-A crystallizer 30 is smaller than the adduct crystallizer 22, and is smaller than crystallizers used in the prior art, since the feed to the Bisphenol-A crystallizer 30 is a mixture of adduct and solvent. The solvent composition may range from about 40 wt % Acetone and 60 wt % Water, to about 60 wt % Acetone and 40 wt % Water. In one exemplary embodiment the solvent composition is comprised of about 51.8 wt. % Acetone and about 48.2 wt. % Water as the adduct exhibits high solubility in this solvent mixture.

As the second crystallization step takes place, solid Bisphenol-A crystals form. The residence time in the Bisphenol-A crystallizer stage 30 is typically in the range of about 1 to 10 hours, more preferably in the range of about 2 to 5 hours. The Bisphenol-A crystals are separated from the mother liquor in the second S/L separator 32, and the mother liquor is preferably recycled to the solvent recovery unit 40. The Bisphenol-A crystals are washed preferably with the solvent in S/L separator 32 to remove any residual solvent. The BPA crystals are then dried in a suitable drying unit (not shown). Further purification steps may be employed if desired. These include re-dissolution of the BPA crystals in a suitable solvent, re-crystallization at a suitable temperature, followed by washing and drying, in order to obtain the desired purity.

It should be made clear that although the above description of the present invention describes crystallization primarily by cooling, the present invention may employ other types of crystallization methods such as evaporative crystallizes, and the like.

### Experimental

Simulated experiments are provided below to further illustrate the system and method of the present invention. These simulated experiments are provided for illustration purposes only and are not intended to limit the scope of the invention in any way.

### EXAMPLE 1

For the simulated experiments, the following design basis was used: 70,000 MTA Bisphenol-A (9,112 kg/hr); Acetone conversion in the condensation reaction of Phenol with Acetone is about 88.5%; Phenol to Acetone feed molar ratio to the condensation reactor is 10; Bisphenol-A mass recovery from adduct crystallizer is about 70%; and Bisphenol-A recovery from the Bisphenol-A crystallizer is about 30%.

In one example, the system units are as follows: The condensation reactor is a fixed type reactor having a size of about 200 M³ at a flow rate of about 77 M³/hr. The condensation reaction is carried out at a temperature of about 75° C., and at a reaction pressure of about 4.4 bar.

In this example, the adduct crystallizer is a circulating Magma Crystallizer with draft-tube-baffle (DTB), having a size of about 150 M³ and receives a flow rate of about 84 M³/hr. The crystallizer is at a temperature of about 51° C. to achieve of Bisphenol-A recovery of about 70%. The temperature can be lowered slightly to increase the Bisphenol-A recovery. The crystallizer and operates at a pressure of about 5 bar. The cooling duty is 1.2 mmKcal/hr for latent heat, and 0.4 mmKcal/hr for heat of fusion.

In this example, the solid/liquid separator (S/L) is a rotary pressure drum filter having a liquid flow rate of about 76,325 kg/hr (excluding the wash liquor) and has a solid flow rate of about 15,161 kg/hr. The S/L is operated at a temperature of about 51° C. and at a pressure of about 5 bar. Preferably the unit is gas tight due to the presence of Acetone in the feed.

A dissolution tank is provided as a mixing tank with a heater. In the exemplary embodiment the tank has a size of about 0.53 M³ and provides a residence time of about 0.3 hours. Of particular advantage the tank is smaller that prior art systems due to the use of Acetone. The adduct exhibits good solubility in the Acetone. Preferably the tank is operated at a temperature of about 115° C. and a pressure of about 5 bar. In this example the liquid flow rate is about 3,499 kg/hr and the solid flow rate is about 15,161 kg/hr.

The Bisphenol-A crystallizer is a circulating Magma Crystallizer with DTB in this example, at a size of about 55 M³ and is operated at a temperature of about 101° C. and a pressure of about 5 bar. The Bisphenol-A crystallizer receives the feed at a flow rate of about 60 M³/hr in this example. The cooling duty of the Bisphenol-A crystallizer is about 0.3 mmKcal/hr for the heat of fusion. Of particular advantage the Bisphenol-A crystallizer is only about 35% of the size of the adduct crystallizer due to the high solution of adduct in the mixture of Acetone and Water.

To separate the Bisphenol-A crystals from the mother liquor following the Bisphenol-A crystallizer, a centrifuge may be employed. Preferably a screen bowl centrifuge is used. In this example the centrifuge receives a liquid flow rate of about 9,534 kg/hr and a solid flow rate of about 9,126 kg/hr. The centrifuge is typically operated at a temperature of about 75° C. and a pressure of about 5 bar.

In the event washing is needed, the amount of washing is dependent upon the quality of the Bisphenol-A product in terms of impurities on the Bisphenol-A crystal surface. If re-crystallization is needed to improve the purity of the Bisphenol-A crystals, it is preferred to use either Acetone or a mixture of Acetone and Water as the solvent because the solubility of Bisphenol-A in Acetone is high and hence the size of the crystallizer and dissolution tank can be reduced.

### EXAMPLE 2

Simulated examples showing typical stream compositions for the major solution streams are provided, corresponding to the present invention as shown in FIG. 6, using two crystallizer stages, the adduct crystallizer stage (Cryst. 1) and the Bisphenol-A crystallizer stage (Cryst. 2). Solution stream compositions for the key process streams—reactor feed and outlet streams, and the crystallizer feed and mother liquor (M/L) streams—are shown in Table 1 below and are in wt %, on an impurity free basis. Note, that impurities are typically present at the inlet and outlet of the reactor, however they are considered to be minimal. Examples of typical impurities, include but are not limited to: by-products such as 2,4-Bisphenol-A, trisphenol, chromans, IPP dimers, and other higher condensation products. In this example, the solvent is comprised of a mixture of two solvent components; namely, Acetone which is a good solvent for Bisphenol-A, and Water which is a poor solvent for Bisphenol-A. The overall Phenol to Acetone ratio in the fresh feed is two (2) on a mole basis.

TABLE 1

| | Reactor | Reactor | Feed to | M/L from | Feed to | M/L from |
|---|---|---|---|---|---|---|
| Compo- | Feed | Outlet | Cryst. 1 | Cryst. 1 | Cryst. 2 | Cryst. 2 |
| nent | wt. % | wt. % | wt. % | wt. % | wt. % | wt. % |
| Phenol | 84.65 | 70.36 | 71.51 | 87.37 | 24.34 | 30.90 |
| BPA | 9.70 | 27.14 | 27.58 | 11.38 | 70.80 | 62.93 |
| Water | 5.22 | 0.60 | 0.43 | 0.59 | 2.52 | 3.20 |
| Acetone | 0.42 | 1.89 | 0.48 | 0.66 | 2.34 | 2.97 |

As shown, the composition of each feed stream is selectively provided such that adduct is formed in Cryst. 1, and pure Bisphenol-A crystals are formed in Cryst. 2.

### EXAMPLE 3

The solution stream compositions shown in the previous example are for a chosen set of operating parameters. In practice, the process has a number of process operating variables, or “handles” which can be changed to vary the process performance. Markedly different process stream compositions can be obtained when the same process is carried out with a different choice of the process handles. For the the process configuration depicted in FIG. 6 the process handles include, but are not limited to: the amount of catalyst in the reactor unit 18; the flow velocity of the reactants through the reactor unit; the Phenol to Acetone ratio at the reactor inlet; the fraction of Solvent recovered by the solvent recovery unit 40; the relative fraction of recovered solvent recycled to the adduct crystallizer 22, the dissolver 28, and the BPA wash unit 32; the fraction of Phenol removed by the solvent recovery unit 40; and the operation temperature, the cooling rate, or the evaporation rate in the Adduct crystallizer 22 and the Bisphenol-A crystallizer 36.

A summary of the process operating at several different solvent environments in the two crystallizers is given below in Table 2. The solvent content and its composition in the Adduct and BPA crystallizers are summarized. As an illustration of the impurity inclusion, the amount of Phenol inclusion in the BPA product is noted. This of course depends process parameters such as the solvent composition, the washing ratio and the efficiency in the washing operation. For typical process operation conditions, less than 10 ppm of Phenol is obtained in the BPA product crystals. Also, the load on the solvent recovery system obtained for different solvent environments in the two crystallizers is noted in the table below.

TABLE 2

| | | | | | | | | Load on |
|---|---|---|---|---|---|---|---|---|
| | Adduct Crystallizer | | | BPA Crystallizer | | | | separation |
| | | Solvent compo- | | | Solvent compo- | | Phenol in | system |
| | Solvent | sition wt. % | | Solvent | sition wt. % | | BPA product | (arbitrary |
| Case | content wt % | Acetone | Water | content wt % | Acetone | Water | (ppm) | units) |
| Case 1 | 0.00 | — | — | 12.02 | 99.93 | 0.07 | 8.52 | 105623 |
| Case 2 | 0.00 | — | — | 6.34 | 51.83 | 48.17 | 9.04 | 103274 |
| Case 3 | 1.25 | 0.60 | 99.40 | 6.23 | 51.80 | 48.20 | 9.05 | 103204 |
| Case 4 | 0.00 | — | — | 6.37 | 76.31 | 23.69 | 9.25 | 102494 |
| Case 5 | 0.00 | — | — | 4.28 | 0.00 | 100.00 | 9.03 | 103320 |
| Case 6 | 1.60 | 1.41 | 98.59 | 6.23 | 51.79 | 48.21 | 9.03 | 103320 |
| Case 7 | 1.25 | 47.23 | 52.77 | 6.17 | 51.83 | 48.17 | 9.03 | 95126 |

In summary, it is shown that the present invention provides a powerful tool wherein numerous routes, system design and operating conditions can be manipulated for selective control of the phase equilibrium diagram. Process variables that can be manipulated to achieve a desired target include: composition of solvent; adding or subtracting solvent and other components in order to change the phase behavior, control of the condensation reaction coupled with control of the process operation conditions to bring the feed composition at the crystallizer to the desired region on the phase diagram; an alteration in the sequence of process operations to achieve the same target; and controlling the recovery of solid in the crystallizer by controlling the temperature once the feed location in the phase diagram region is fixed. Further, according to the system configuration of the present invention, no separation of Water and Acetone from the product stream is required after the reactor (and before the adduct crystallization).

Of particular advantage, the system and process of the present invention does not require expensive steps shown in the prior art for the complete evaporation of Phenol from the Adduct. As discussed above, conventional prior art methods of recovering Bisphenol-A from the adduct require melting the adduct and removing Phenol by evaporation for example in a wiped film evaporator, and the recovering Bisphenol-A by melt or solution crystallization. Following this, solid Bisphenol-A product is obtained from molten Bisphenol-A by a prilling process. In the present invention, only a partial vaporization of Phenol takes place in the Solvent recovery, said vaporization being much cheaper because it is carried out at a much higher concentration of Phenol, than the evaporation of Phenol to the “last drop” carried out in the prior art method. Furthermore, in the present invention, Bisphenol-A is recovered directly in the crystal form in the second crystallizer stage, and thus the need for a prilling tower is eliminated, providing further capital cost and energy savings.

The foregoing description of specific embodiments and examples of the invention have been presented for the purpose of illustration and description, and although the invention has been illustrated by certain of the preceding examples, it is not to be construed as being limited thereby. They are not intended to be exhaustive or to limit the invention to the precise forms disclosed, and obviously many modifications, embodiments, and variations are possible in light of the above teaching. It is intended that the scope of the invention encompass the generic area as herein disclosed, and by the claims appended hereto and their equivalents.

1. A method of producing Bisphenol-A, comprising the steps of:
reacting Phenol and Acetone in the presence of a catalyst to form a product solution including at least Bisphenol-A and Phenol, and where the Bisphenol-A, Phenol and a solvent exhibit a phase equilibrium relationship between the components of the product solution such that they exhibit compartments represented by a projection of a polythermal ternary phase diagram where Bisphenol-A and an adduct of Bisphenol-A and Phenol can crystallize respectively;
providing the solvent in said product solution, wherein said solvent is comprised of Acetone, Water or a mixture of Acetone and Water;
crystallizing an adduct of Bisphenol-A and Phenol from a first crystallization stage at a first selective composition of a feed stream to an adduct crystallizer stage such that the adduct is formed;
dissolving the adduct in a solvent having a selective composition; and
crystallizing substantially pure solid Bisphenol-A in a second crystallization stage at a second selective composition of a feed stream to a Bisphenol-A crystallizer stage such that the substantially pure Bisphenol-A is crystallized.

2. The method of claim 1 wherein the first and second selective compositions of feed solutions are achieved by adjusting the mass concentration of any one of, or combination of, Phenol, Bisphenol-A and solvent in the feed streams.

3. The method of claim 1 wherein the fisrst selective composition of the feed stream to the adduct crystallizer is comprised of Bisphenol-A at a composition in a range of approximately 40 wt. % to 60 wt. %, and a solvent at a composition in a range of approximately 0 wt. % to 15 wt. %.

4. The method of claim 1 wherein the fisrst selective composition of the feed stream to the adduct crystallizer is comprised of Bisphenol-A at a composition in a range of approximately 25 wt. % to 60 wt. %, and a solvent at a composition in a range of approximately 0 wt. % to 25 wt. %.

5. The method of claim 1 wherein the fisrst selective composition of the feed stream to the adduct crystallizer is comprised of Bisphenol-A at a composition in a range of approximately 10 wt. % to 60 wt. %, and a solvent at a composition in a range of approximately 0 wt. % to 35 wt. %.

6. The method of claim 1 wherein the second selective composition of the feed stream to the Bisphenol-A crystallizer is comprised of Bisphenol-A at a composition in a range of approximately 55 wt. % to 100 wt. %, and a solvent at a composition in a range of approximately 0 wt. % to 15 wt. %.

7. The method of claim 1 wherein the second selective composition of the feed stream to the Bisphenol-A crystallizer is comprised of Bisphenol-A at a composition in a range of approximately 55 wt. % to 100 wt. %, and a solvent at a composition in a range of approximately 0 wt. % to 40 wt. %.

8. The method of claim 1 wherein the first stage crystallization is carried out at a temperature in the range of about 0° C. to 100° C.

9. The method of claim 1 wherein the first stage crystallization is carried out at a temperature in the range of about 20° C. to 80° C.

10. The method of claim 1 wherein the first stage crystallization is carried out at a temperature in the range of about 40° C. to 60° C.

11. The method of claim 1 wherein the second stage crystallization is carried out at a temperature in the range of about 0° C. to 160° C.

12. The method of claim 1 wherein the second stage crystallization is carried out at a temperature in the range of about 80° C. to 120° C.

13. The method of claim 1 wherein the solvent is comprised of a mixture of about 50% Acetone and about 50% Water on a weight basis.

14. The method of claim 1 wherein the solvent is comprised of a mixture Acetone and Water at a composition in the range of 99 wt. % Acetone and 1 wt. % Water to 1 wt. % Acetone and 99 wt. % Water.

15. The method of claim 1 wherein the solvent is comprised of a mixture of Acetone and Water at a composition in the range of 40 wt. % Acetone and 60 wt. % Water to 60 wt. % Acetone and 40 wt. % Water.

16. A method of recovering Bisphenol-A solid from an adduct of Bisphenol-A and Phenol, comprising the steps of:
dissolving the adduct in a solvent to form a feed solution; and
crystallizing Bisphenol-A from the feed solution wherein the composition of the feed solution and the temperature of the crystallizing step are selected such that substantially pure Bisphenol-A solid is crystallized.

17. A method of producing solid Bisphenol-A from a product solution, characterized in that the product solution includes components that exhibit a phase equilibrium relationship they define phase compartments represented by a projection of a polythermal ternary phase diagram, where Bisphenol-A and an adduct of Bisphenol-A and Phenol can crystallize respectively; and the composition of the product stream is selected to be within the Bisphenol-A phase compartment such that substantially pure solid Bisphenol-A is formed upon crystallization.

18. A system for producing Bisphenol-A comprising:
a reactor unit wherein a product stream is produced including at least Bisphenol-A and Phenol;
a first stage crystallizer, the composition of the product stream fed to the first stage crystallizer being selectively adjusted such that an adduct of Bisphenol-A and Phenol is produced upon crystallization in the first stage crystallizer;
a dissolution tank where the adduct of Bisphenol-A and Phenol is dissolved in the product stream; and
a second stage crystallizer, the second stage crystallizer receiving the product stream and the composition of the product stream is selectively adjusted such that substantially pure Bisphenol-A is produced upon crystallization in the second stage crystallizer.

19. The system of claim 18 further comprising:
a first mixer/separator unit located upstream of the first stage crystallizer, the mixer/separator unit configured to selectively adjust the composition of the product stream fed to the first stage crystallizer.

20. The system of claim 19 further comprising:
a solvent recovery unit for recovering solvent used in the system, and said solvent recovery unit being coupled to at least any one or more of the reactor unit, dissolution tank and mixer/separator for selectively adjusting the composition of the product stream.

21. The system of claim 18 where in the first and second crystallizer stages are comprised of one or more crystallizer unit.

22. The system of claim 18 further comprising:
a second mixer/separator unit located upstream of the second stage crystallizer, the mixer/separator unit being configured to selectively adjust the composition of the product stream fed to the second stage crystallizer.

23. The system of claim 22 further comprising one or more processing units downstream of the second stage crystallizer, and wherein the one or more processing units produce recycle streams, and where one or more of the recycle streams are conveyed to the first and/or second mixer/separator.
